(19) United States
(12) Patent Application Publication
(10) Pub. No.: US 2022/0227709 A1
(43) Pub. Date: Jul. 21, 2022

(54) COMPOSITIONS AND METHODS FOR TREATING SCHISTOSOMA INFECTIONS

(71) Applicant: THE BOARD OF REGENTS OF THE UNIVERSITY OF TEXAS SYSTEM, Austin, TX (US)

(72) Inventors: Stanton F. McHardy, Waring, TX (US); Philip T. Loverde, San Antonio, TX (US); TIM JC ANDERSON, San Antonio, TX (US); REID SAMUEL TARPLEY, San Antonio, TX (US); ALEXANDER BRYAN TAYLOR, San Antonio, TX (US)

(21) Appl. No.: 17/607,505

(22) PCT Filed: Apr. 29, 2020
(86) PCT No.: PCT/US2020/030503

§ 371 Date: Oct. 29, 2021

Related U.S. Application Data
(60) Provisional application No. 62/840,362, filed on Apr. 29, 2019.

(51) Int. Cl. C07D 207/09; C07D 403/06; A61K 31/401; A61K 31/404; A61P 33/12; A61K 31/4985

(52) U.S. Cl. CPC C07D 207/09; C07D 403/06; A61K 31/4985; A61K 31/404; A61P 33/12; A61K 31/401

(57) ABSTRACT

Disclosed are composition and methods for treating Schistosoma infection.

### CROSS REFERENCE TO RELATED APPLICATIONS

This Application claims priority to U.S. Provisional Application Ser. No. 62/840,362 filed Apr. 29, 2019, which is incorporated herein by reference in its entirety.

### FIELD OF THE INVENTION

The present invention relates generally to the field of medicine. More particularly, it concerns compositions and methods for treating Schistosoma infections.

### BACKGROUND

Schistosomiasis is endemic in 74 developing countries with more than 80% of infected people living in sub-Saharan Africa. The causative agent is a trematode flatworm (fluke) of the genus Schistosoma. S. haematobium is a prevalent and widespread species in Africa and the Middle East. S. intercalatum occurs in 10 countries in the rainforest belt of Africa. S. mansoni is found in Africa, and S. japonicum is found in the Pacific region including China and the Philippines. S. mekongi is found in limited areas of Laos and Cambodia.

Transmission occurs in freshwater when intermediate snail hosts release infective forms of the parasite. People are infected by contact with water where infected snails live. Larval forms of the parasites (known as cercariae), are released by the snails, and penetrate the skin of people in the water. The snails themselves become infected by another larval stage of the parasite, known as a miracidium, which develops from eggs passed out in the urine or feces of infected people.

Adult male and female Schistosomes pair and live together in human blood vessels. The females release eggs, some of which are passed out in the urine or stools, but some eggs are trapped in body tissues. Some aspects of the disease are caused by immune reactions to eggs lodged in tissues. In urinary schistosomiasis, damage to the urinary tract is revealed by blood in the urine. Urination becomes painful and is accompanied by progressive damage to the bladder, ureters and then the kidneys. Bladder cancer may occur in advanced cases. In intestinal schistosomiasis (e.g., as a result of infection with S. mansoni, S. japonicum) disease is slower to develop. There is progressive enlargement of the liver and spleen, intestinal damage due to fibrotic lesions around eggs lodged in these tissues, and hypertension of the abdominal blood vessels. Bleeding from these vessels leads to blood in stools, and can be fatal. Functioning of organs such as spleen and kidneys becomes impaired. Bleeding from varicose veins in the esophagus is associated with intestinal schistosomiasis. Children are especially vulnerable to infection, which develops into chronic disease if not treated.

There remains a need for additional treatments for Schistosoma infections

### SUMMARY OF THE DISCLOSURE

It has been determined that compounds having a chemical structure of Formula I, Formula II, or a pharmaceutically acceptable salt or solvate thereof, can be effective in treating Schistosoma infections.

Certain embodiments are directed to a 3,3′-disubstituted pyrrolidine derivatives having a chemical structure of Formula I

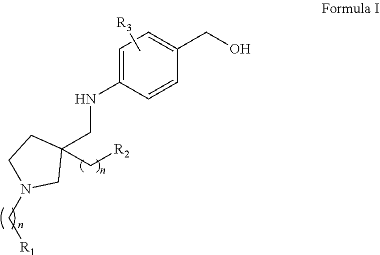

wherein n is 0 or 1; R₁ and R₂ can be independently selected from H; alkyl; branched alkyl; substituted alkyl; aryl; benzene; 1-, 2-, or 3-fluorobenzene; substituted or unsubstituted indole; substituted or unsubstituted indazole; 1-, 2-, or 3-trifluoromethylbenzene; 1-, 2-, or 3-trifluoromethoxybenzene; fluoro-trifluoromethylbenzene; difluoromethylbenzene; and —CH₂—Ar. R₃ can be F; Cl; Br; I; —NO₂; CO₂H; —CN; —CF₃; CO₂—R₁; CONH₂; CONHR₄; or CON(R₁)₂. In certain aspects, the R₃ substituent is at the 1, or 2 position. In some aspects, R₁=CH₂-1H-indol-3-yl)methyl, R₂=3-trifluoromethylbenzyl, and R₃=NO₂. In some aspects, wherein R₁=CH₂C₆H₅, R₂=3-trifluoromethylbenzyl, and R₃=NO₂. In some aspects, wherein R₁=H, R₂=3-trifluoromethylbenzyl, and R₃=NO₂. In certain aspect, an indazole can be a 1-, 2-, or 3-methylindozole and in particular a 1-methylindazole.

Certain embodiments are directed to a compound having a chemical structure of Formula II

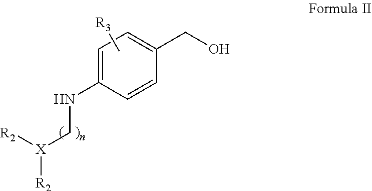

wherein X is piperidine or a pyrrolidine; n is 0 or 1; R₁ and R₂ can be independently selected from H; alkyl; branched alkyl; substituted alkyl; aryl; benzene; 1-, 2-, or 3-fluorobenzene; 1-, 2-, or 3-trifluoromethylbenzene; 1-, 2-, or 3-trifluoromethoxybenzene; fluoro-trifluoromethylbenzene; difluoromethylbenzene; substituted or unsubstituted indole; substituted or unsubstituted indazole; and —CH₂—Ar; and R₃ can be F; Cl; Br; I; —NO₂; CO₂H; —CN; —CF₃; CO₂—R₁; CONH₂, CONHR₁, or CON(R₁)₂. In certain aspects, the R2 substituent is at the 2 position of the pyrrolidine or piperidine and the R1 substituent is bound to the nitrogen od the pyrrolidine or piperidine. In some aspects, R₁=CH₂-1H-indol-3-yl)methyl, R₂=3-trifluoromethylbenzyl, and R₃=NO₂. In some aspects, wherein R₁=CH₂C₆H₅, R₂=3-trifluoromethylbenzyl, and R₃=NO₂. In some aspects, wherein R₁=H, R₂=3-trifluoromethylbenzyl, and R₃=NO₂.

Certain embodiments are directed to any 1, 2, 3, 4, 5, 6, 7, 8, 9, 10, 11, 12, 13, 14, 15, 16, 17, 18, 19, 20 or more of (2-nitro-4-(((3-(3-(trifluoromethyl)benzyl)pyrrolidin-3-yl)methyl)amino)phenyl)methanol; (4-(((1-((1H-indol-3-yl)methyl)-3-(3-(trifluoromethyl)benzyl)pyrrolidin-3-yl)methyl)amino)-2-nitrophenyl)methanol; (4-(((1-benzyl-3-(3-(trifluoromethyl)benzyl)pyrrolidin-3-yl)methyl)amino)-2-nitrophenyl)methanol; (R)-(4-(((1-((1H-indol-3-yl)methyl)-3-(3-(trifluoromethyl)benzyl)pyrrolidin-3-yl)methyl)amino)-2-nitrophenyl)methanol; (S)-(4-(((1-((1H-indol-3-yl)methyl)-3-(3-(trifluoromethyl)benzyl)pyrrolidin-3-yl)methyl)amino)-2-nitrophenyl)methanol; (R)-(2-nitro-4-((1-(3-(trifluoromethyl)benzyl)piperidin-3-yl)amino)phenyl)methanol; (4-(((1-((1-methyl-1H-indol-3-yl)methyl)-3-(3- trifluoromethyl)benzyl)pyrrolidin-3-yl)methyl)amino)-2-nitrophenyl)methanol; (4-(((1-((1H-indazol-3-yl)methyl)-3-(3-(trifluoromethyl)benzyl)pyrrolidin-3-yl)methyl)amino)-2-nitrophenyl)methanol; (4-(((1-((1H-pyrrolo[2,3-b]pyridin-3-yl)methyl)-3-(3-(trifluoromethyl)benzyl)pyrrolidin-3-yl)methyl)amino)-2-nitrophenyl)methanol; (4-(((1-((1-methyl-1H-indol-2-yl)methyl)-3-(3-(trifluoromethyl)benzyl)pyrrolidin-3-yl)methyl)amino)-2-nitrophenyl)methanol; (4-(((1-(indolin-7-ylmethyl)-3-(3-(trifluoromethyl)benzyl)pyrrolidin-3-yl)methyl)amino)-2-nitrophenyl)methanol; (4-(((1-((1H-indol-6-yl)methyl)-3-(3-(trifluoromethyl)benzyl)pyrrolidin-3-yl)methyl)amino)-2-nitrophenyl)methanol; (4-(((1-((2-methyl-2H-indazol-3-yl)methyl)-3-(3-(trifluoromethyl)benzyl)pyrrolidin-3-yl)methyl)amino)-2-nitrophenyl)methanol; (4-(((1-((2H-indol-3-yl)methyl)-3-(3-(trifluoromethyl)benzyl)pyrrolidin-3-yl)methyl)amino)-2-nitrophenyl)methanol; (R)-(3-nitro-4-(piperidin-3-ylamino)phenyl)methanol; (S)-(3-nitro-4-(piperidin-3-ylamino)phenyl)methanol; (4-((1-((1H-indol-3-yl)methyl)piperidin-3-yl)amino)-3-nitrophenyl)methanol; (3-nitro-4-((1-(2-(trifluoromethoxy)benzyl)piperidin-3-yl)amino)phenyl)methanol; (2-nitro-4-(((1-(2-(trifluoromethyl)benzyl)-3-(3-(trifluoromethyl)benzyl)pyrrolidin-3-yl)methyl)amino)phenyl)methanol; (4-(((1,3-bis(3-(trifluoromethyl)benzyl)pyrrolidin-3-yl)methyl)amino)-2-nitrophenyl)methanol; (2-nitro-4-(((3-(3-(trifluoromethyl)benzyl)-1-(4- trifluoromethyl)benzyl)pyrrolidin-3-yl)methyl)amino)phenyl)methanol; (2-nitro-4-(((1-(2-(trifluoromethoxy)benzyl)-3-(3-(trifluoromethyl)benzyl)pyrrolidin-3-yl)methyl)amino)phenyl)methanol; (2-nitro-4-(((1-(3-(trifluoromethoxy)benzyl)-3-(3-(trifluoromethyl)benzyl)pyrrolidin-3-yl)methyl)amino)phenyl)methanol; (2-nitro-4-(((1-(4-(trifluoromethoxy)benzyl)-3-(3-(trifluoromethyl)benzyl)pyrrolidin-3-yl)methyl)amino)phenyl)methanol; (4-(((1-(2-fluorobenzyl)-3-(3-(trifluoromethyl)benzyl)pyrrolidin-3-yl)methyl)amino)-2-nitrophenyl)methanol; (4-(((1-(3-fluorobenzyl)-3-(3-(trifluoromethyl)benzyl)pyrrolidin-3-yl)methyl)amino)-2-nitrophenyl)methanol; (4-(((1-(4-fluorobenzyl)-3-(3-(trifluoromethyl)benzyl)pyrrolidin-3-yl)methyl)amino)-2-nitrophenyl)methanol; (4-((1-(2-fluoro-4-(trifluoromethyl)benzyl)piperidin-3-yl)amino)-3-nitrophenyl)methanol; (3-nitro-4-((1-(3-(trifluoromethoxy)benzyl)piperidin-3- yl)amino)phenyl)methanol; (3-nitro-4-((1-(3-(trifluoromethyl)benzyl)piperidin-3-yl)amino)phenyl)methanol; (S)-5-(hydroxymethyl)-2-((1-(2-(trifluoromethyl)benzyl)piperidin-3-yl)amino)benzonitrile; (4-((1-(2-(difluoromethyl)benzyl)piperidin-3-yl)amino)-3-nitrophenyl)methanol; (4-((1-((1H-indol-2-yl)methyl)piperidin-3-yl)amino)-3-nitrophenyl)methanol; (4-((1-((1-methyl-1H-indol-3-yl)methyl)piperidin-3-yl)amino)-3-nitrophenyl)methanol; (4-((1-((1-methyl-1H-indol-2-yl)methyl)piperidin-3-yl)amino)-3-nitrophenyl)methanol; (R)-(4-((1-(2-(difluoromethyl)benzyl)piperidin-3-yl)amino)-2-nitrophenyl)methanol; (4-(((1-benzyl-3-(2-(trifluoromethyl)benzyl)pyrrolidin-3-yl)methyl)amino)-2- nitrophenyl)methanol; (4-(((1-((1H-indol-3-yl)methyl)-3-(2-(trifluoromethyl)benzyl)pyrrolidin-3-yl)methyl)amino)-2-nitrophenyl)methanol; (4-(((1-benzyl-3-(2-(trifluoromethyl)benzyl)pyrrolidin-3-yl)methyl)amino)-3-nitrophenyl)methanol; (4-(((1-((1H-indol-3-yl)methyl)-3-(2-(trifluoromethyl)benzyl)pyrrolidin-3-yl)methyl)amino)-3-nitrophenyl)methanol; (4-(((1-benzyl-3-(4- (trifluoromethyl)benzyl)pyrrolidin-3-yl)methyl)amino)-2-nitrophenyl)methanol; (4-(((1-((1H-indol-3-yl)methyl)-3-(4-(trifluoromethyl)benzyl)pyrrolidin-3-yl)methyl)amino)-2-nitrophenyl)methanol; (4-(((1-benzyl-3-(4-(trifluoromethyl)benzyl)pyrrolidin-3-yl)methyl)amino)-3-nitrophenyl)methanol; and/or (4-(((1-((1H-indol-3-yl)methyl)-3-(4-(trifluoromethyl)benzyl)pyrrolidin-3-yl)methyl)amino)-3-nitrophenyl)methanol. In certain aspects and one or more of these compounds can be specifically excluded.

Certain embodiments are directed to compositions and methods of use for a compound having a chemical structure of Formula I or a pharmaceutically acceptable salt or solvate thereof. Certain embodiments are directed to a pharmaceutical composition containing an effective amount of a compound having a chemical structure of Formula I or a pharmaceutically acceptable salt or solvate thereof. In some aspects, the pharmaceutical composition can contain one or more of the compounds illustrated in FIG. 1-3 or a pharmaceutically acceptable salt or solvate thereof. In some aspects, the pharmaceutical composition can contain a pharmaceutically acceptable carrier. In some embodiments the composition can further contain an effective amount of praziquantel. In some aspects, the pharmaceutical composition can contain a pharmaceutically acceptable excipient. In some embodiments, one or more compounds having a chemical structure of Formula I can be excluded. In some embodiments, one or more of the compounds illustrated in FIG. 1-3 may be excluded.

Certain embodiments are directed to a method of treating Schistosoma infections the method includes administering an effective amount of a compound having a chemical structure of Formula I or a pharmaceutically acceptable salt or solvate thereof, to a subject in need thereof. In some aspects, one or more of the compounds illustrated in FIG. 1-3 or a pharmaceutically acceptable salt or solvate thereof is administered. In some embodiments the method further includes administering an effective amount of praziquantel. Praziquantel before, after and/or during with administering of a compound having a chemical structure of Formula I or a pharmaceutically acceptable salt or solvate thereof. Praziquantel and a compound having a chemical structure of Formula I or a pharmaceutically acceptable salt can be administered with a same composition. Praziquantel and a compound having a chemical structure of Formula I or a pharmaceutically acceptable salt can be administered with a different composition. In some aspects, the Schistosoma infection is infection by Schistosoma mansoni, Schistosoma haematobium, Schistosoma japonicum or a combination thereof.

In some aspects, a compound having a chemical structure of Formula I, has the chemical structure provided in FIG. 1 (3,3′-disubstituted pyrrolidine derivative 1, (compound 9)). In some aspects, a compound having a chemical structure of Formula I, has the chemical structure provided in FIG. 2 (3,3′-disubstituted pyrrolidine derivative 2). In some aspects, a compound having a chemical structure of Formula I, has the chemical structure provided in FIG. 3 (3,3′-disubstituted pyrrolidine derivative 3).

In some embodiments, the route of administration is oral, parenteral, subcutaneous or intramuscular. In some embodiments, a compound having a chemical structure of Formula I or a pharmaceutically acceptable salt or solvate thereof is provided over multiple administrations. In some aspects, an effective amount of a compound having the chemical structure provided in FIG. 1 or a pharmaceutically acceptable salt or solvate thereof is administered. In some aspects, an effective amount of a compound having the chemical structure provided in FIG. 2 or a pharmaceutically acceptable salt or solvate thereof is administered. In some aspects, an effective amount of a compound having the chemical structure provided in FIG. 3 or a pharmaceutically acceptable salt or solvate thereof is administered. In some aspects, a combination of the compounds of the disclosure is administered. In some embodiments, one or more of the compounds may be excluded.

Other embodiments are directed to any of the compounds illustrated in FIG. 1-3. In some embodiments, one or more of the compounds illustrated in FIG. 1-3 may be excluded.

A “subject,” “individual” or “patient” is used interchangeably herein and refers to a vertebrate, for example a primate, a mammal or a human. Mammals include, but are not limited to equines, canines, bovines, ovines, murines, rats, simians, humans, farm animals, sport animals, and pets. Also intended to be included as a subject are any subjects involved in clinical research trials not showing any clinical sign of disease, or subjects involved in epidemiological studies, or subjects used as controls. In some aspect the subject is human.

As used herein, the term “comprising” is intended to mean that the compositions and methods include the recited elements, but not excluding others. “Consisting essentially of” when used to define compositions and methods, shall mean excluding other elements of any essential significance to the combination for the stated purpose. “Consisting essentially of” in the context of pharmaceutical compositions of the disclosure is intended to include all the recited active agents and excludes any additional non-recited active agents, but does not exclude other components of the composition that are not active ingredients. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention or process steps to produce a composition or achieve an intended result. Embodiments defined by each of these transition terms are within the scope of this invention.

The terms “ameliorating,” “inhibiting,” or “reducing,” or any variation of these terms, when used in the claims and/or the specification includes any measurable decrease or complete inhibition to achieve a desired result.

As used herein, “treating,” “treatment” or “therapy” is an approach for obtaining beneficial or desired clinical results. This includes: reduce the alleviation of symptoms or any relevant result described throughout the disclosure, including the examples. Furthermore, these terms are intended to encompass curing as well as ameliorating at least one symptom of the condition or disease.

Use of the one or more compositions may be employed based on methods described herein. Use of one or more compositions may be employed in the preparation of medicaments for treatments according to the methods described herein. Other embodiments are discussed throughout this application. Any embodiment discussed with respect to one aspect of the disclosure applies to other aspects of the disclosure as well and vice versa. The embodiments in the Example section are understood to be embodiments that are applicable to all aspects of the technology described herein.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell biology to indicate that a value includes the standard deviation of error for the device or method being employed to determine the value.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

The term “therapeutically effective amount” refers to an amount of the drug that treats or inhibits a disease or condition. In some embodiments, the therapeutically effective amount inhibits at least or at most or exactly 100, 99, 98, 96, 94, 92, 90, 85, 80, 75, 70, 65, 60, 55, 50, 40, 30, 20, or 10%, or any derivable range therein, of the activity or expression of a gene described herein.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: 3,3′-disubstituted pyrrolidine derivative 1 (compound 9).

FIG. 2: 3,3′-disubstituted pyrrolidine derivative 2.

FIG. 3: 3,3′-disubstituted pyrrolidine derivative 3.

FIG. 4: Synthesis of 3,3′-disubstituted pyrrolidine derivative 1 (compound 9).

FIG. 5A-5D: (A-D) Representative chemicals and structures.

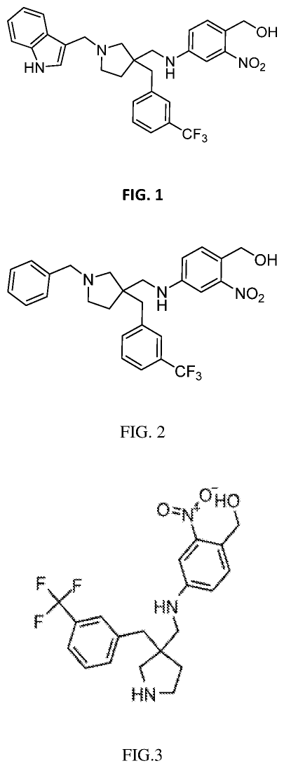

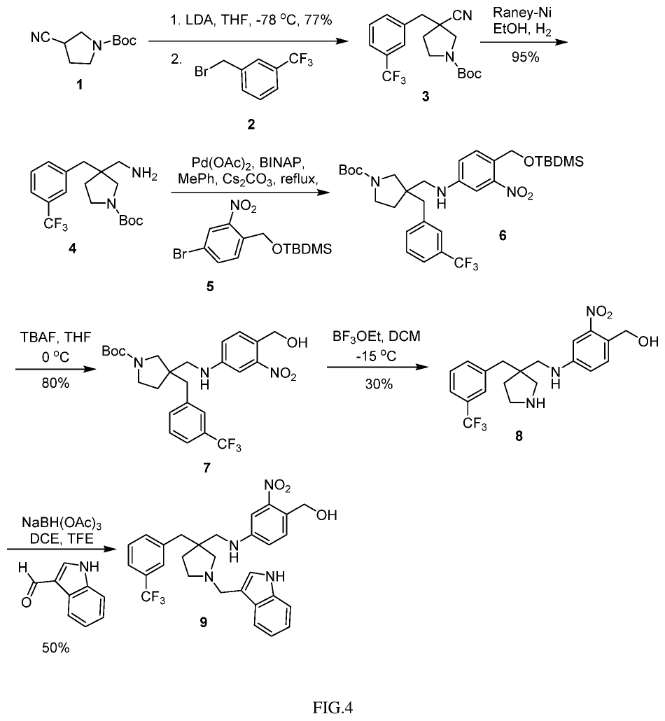

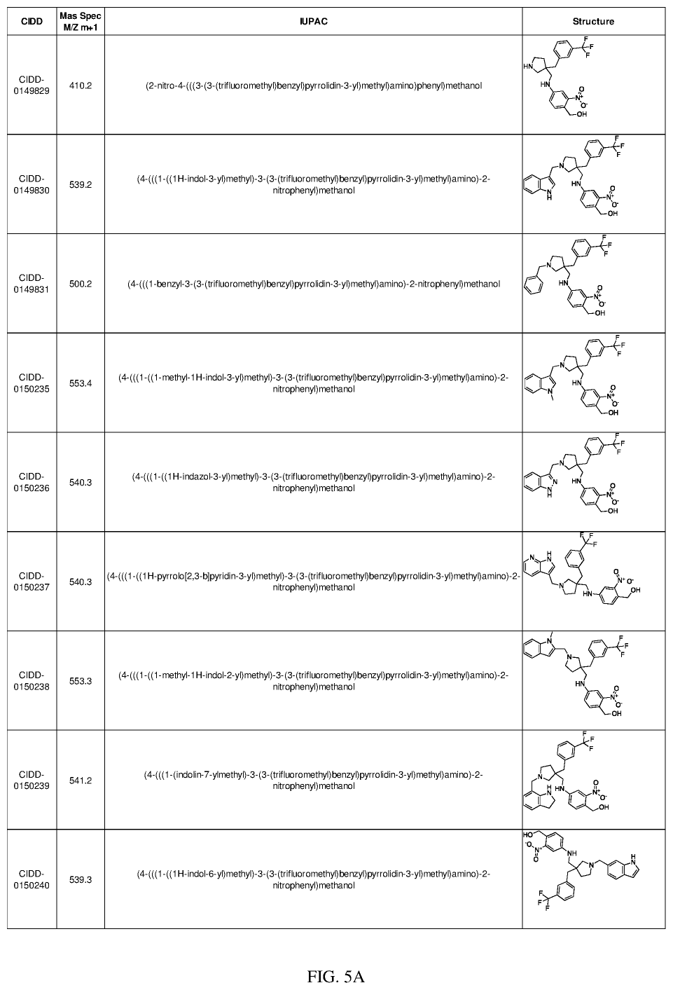

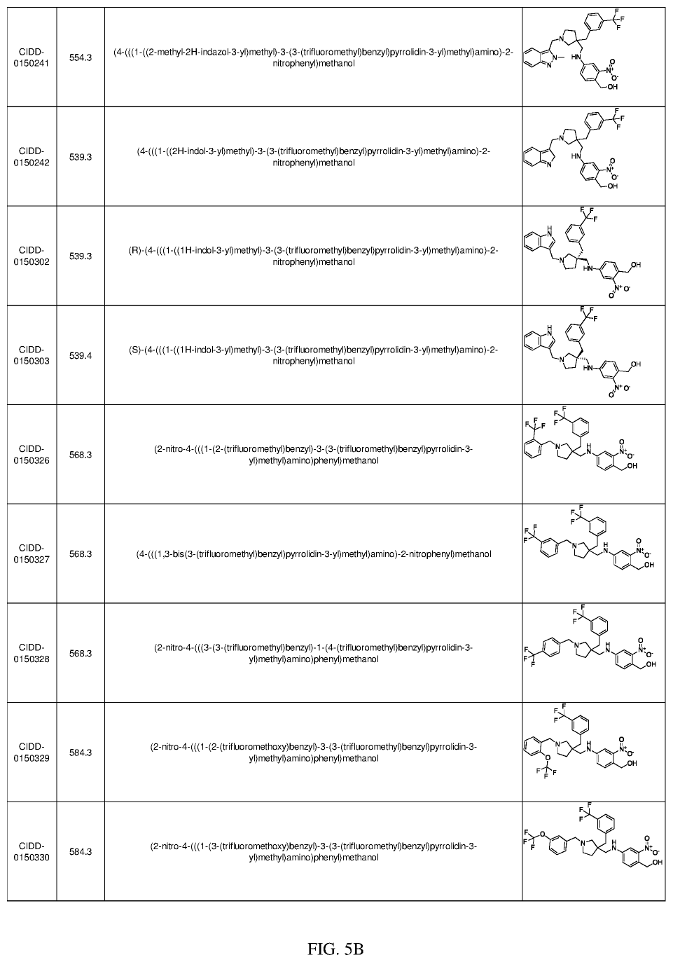

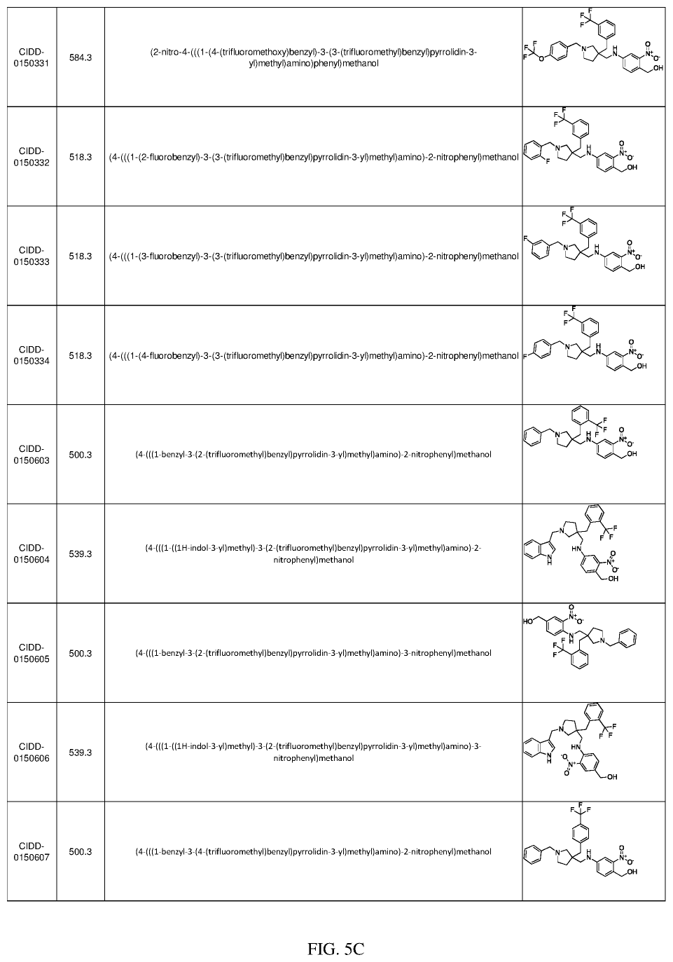

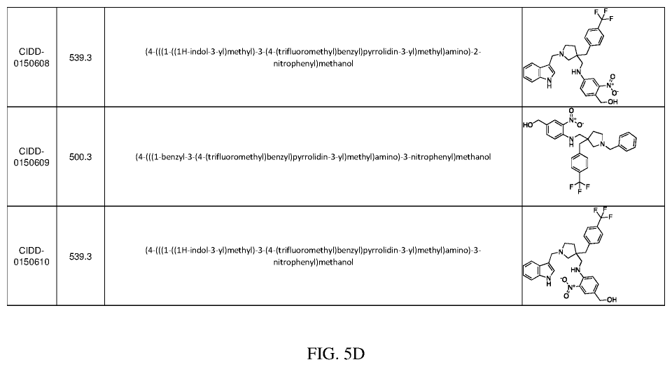

### DESCRIPTION OF ILLUSTRATIVE EMBODIMENTS

It has been determined that compounds having a chemical structure of Formula I or a pharmaceutically acceptable salt or solvate thereof, can be effective in treating Schistosoma infection. In some aspects, the Schistosoma infection can be an infection by one or more of Schistosoma mansoni, Schistosoma haematobium or Schistosoma japonicum

In some embodiments, the subject is administered a composition in which a compound described herein is the active ingredient in the composition.

### I. PHARMACEUTICAL COMPOSITIONS AND METHODS OF ADMINISTRATION

Compositions and methods described herein may involve administering to patients via any route used to introduce therapy to patients. Such routes include, but are not limited to, administration intravenously, intradermally, intraarterially, intraperitoneally, intralesionally, intracranially, intraarticularly, intraprostaticaly, intrapleurally, intratracheally, intranasally, intrathecally, intravitreally, intravaginally, intrarectally, topically, intratumorally, intramuscularly, intraperitoneally, intraocularly, subcutaneously, subconjunctival, intravesicularlly, mucosally, intrapericardially, intraumbilically, intraocularally, orally, topically, locally, by inhalation, by injection, by infusion, by continuous infusion, by localized perfusion, via a catheter, via nebulizer, or via a lavage, or various combinations thereof. In specific embodiments, the composition is administered to the subject by inhalation. In particular embodiments, the composition is administered to the subject as an aerosol. Other examples of routes of administration involve a nebulizer.

The compositions may be formulated in a pharmaceutically acceptable composition. In certain embodiments, a preservative and/or stabilizer is included in the composition.

Furthermore, in some embodiments, compositions may contain about, at least about, or at most about 0.1, 0.2, 0.3, 0.4, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 21, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 ng, μg or mg of a compound of the disclosure (or any range derivable therein), which may be in about, at least about, or at most about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 10, 11, 12, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 μl or ml (or any range derivable therein). Alternatively, a composition may have a concentration of a compound of at least about, at most about, or about 0.1, 0.2, 0.3, 0.4, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 21, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 μM or mM (or any range derivable therein).

Moreover, such amounts may be administered to a subject in one or more doses as that much compound of the disclosure/kg body weight of the subject. For example, a subject may be administered an amount in the range of about 1 μg/kg and about 1 mg/kg. In certain embodiments, the amount given to a subject is about, at least about, or at most about 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 21, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 μg/kg or mg/kg, or any range derivable therein. These amounts may be prescribed on a per administration basis or on a daily basis (for example on a μg/kg body weight/day basis).

Such amounts can be administered daily, though other dosing regimens are contemplated. It is contemplated that compositions may be administered just a single time or multiple times. In certain embodiments, a composition is administered 1, 2, 3, 4, 5, 6, or more times, or any range derivable therein. It is contemplated that a preventative or treatment regimen may involve multiple administrations over 1, 2, 3, 4, 5, 6, and/or 7 days or 1, 2, 3, 4, or 5 weeks, and/or 1, 2, 3, 4, 5, 6, 7, 8, 9, 10, 11, and/or 12 months, or any range derivable therein. Moreover, any such regimen may be repeated after a certain amount of time has passed or when symptoms of the disease or condition become noticeable or more severe.

In certain aspects, the compositions or agents for use in the methods, such as therapeutic compounds, are suitably contained in a pharmaceutically acceptable carrier. The carrier is non-toxic, biocompatible and is selected so as not to detrimentally affect the biological activity of the agent. The agents in some aspects of the disclosure may be formulated into preparations for local delivery (i.e. to a specific location of the body, such as skeletal muscle or other tissue) or systemic delivery, in solid, semi-solid, gel, liquid, or gaseous forms such as tablets, capsules, powders, granules, ointments, solutions, depositories, inhalants, and injections allowing for oral, parenteral, or surgical administration. Certain aspects of the disclosure also contemplate local administration of the compositions by coating medical devices, local administration, and the like.

Suitable carriers for parenteral delivery via injectable, infusion, or irrigation, and topical delivery include distilled water, physiological phosphate-buffered saline, normal or lactated Ringer's solutions, dextrose solution, Hank's solution, or propanediol. In addition, sterile, fixed oils may be employed as a solvent or suspending medium. For this purpose any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste, or salve.

The carrier may also comprise a delivery vehicle to sustain (i.e., extend, delay or regulate) the delivery of the agent(s) or to enhance the delivery, uptake, stability, or pharmacokinetics of the therapeutic agent(s). Such a delivery vehicle may include, by way of non-limiting examples, microparticles, microspheres, nanospheres, or nanoparticles composed of proteins, liposomes, carbohydrates, synthetic organic compounds, inorganic compounds, polymeric or copolymeric hydrogels, and polymeric micelles.

In certain aspects, the actual dosage amount of a composition administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active agent, such as an isolated exosome, a related lipid nanovesicle, or an exosome or nanovesicle loaded with therapeutic agents or diagnostic agents. In other embodiments, the active agent may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 microgram/kg/body weight to about 100 mg/kg/body weight, about 5 microgram/kg/body weight to about 500 milligram/kg/body weight, etc., can be administered.

Solutions of pharmaceutical compositions can be prepared in water suitably mixed with a surfactant, such as hydroxypropylcellulose. Dispersions also can be prepared in glycerol, liquid polyethylene glycols, mixtures thereof, and in oils. Under ordinary conditions of storage and use, these preparations contain a preservative to prevent the growth of microorganisms.

In certain aspects, the pharmaceutical compositions are advantageously administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg or less, 25 mg, 50 mg, or up to about 100 mg of human serum albumin per milliliter of phosphate buffered saline. Other pharmaceutically acceptable carriers include aqueous solutions, non-toxic excipients, including salts, preservatives, buffers, and the like.

Examples of non-aqueous solvents are propylene glycol, polyethylene glycol, vegetable oil, and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, antgifungal agents, anti-oxidants, chelating agents and inert gases. The pH and exact concentration of the various components the pharmaceutical composition are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate, and the like. The compositions take the form of solutions, suspensions, tablets, pills, capsules, sustained release formulations, or powders.

In further aspects, the pharmaceutical compositions may include classic pharmaceutical preparations. Administration of pharmaceutical compositions according to certain aspects may be via any common route so long as the target tissue is available via that route. This may include oral, nasal, buccal, rectal, vaginal, or topical. Alternatively, administration may be by orthotopic, intradermal, subcutaneous, intramuscular, intraperitoneal or intravenous injection. Such compositions would normally be administered as pharmaceutically acceptable compositions that include physiologically acceptable carriers, buffers, or other excipients. For treatment of conditions of the lungs, aerosol delivery can be used. Volume of the aerosol is between about 0.01 ml and 0.5 ml.

An effective amount of the pharmaceutical composition is determined based on the intended goal. The term “unit dose” or “dosage” refers to physically discrete units suitable for use in a subject, each unit containing a predetermined-quantity of the pharmaceutical composition calculated to produce the desired responses discussed above in association with its administration, i.e., the appropriate route and treatment regimen. The quantity to be administered, both according to number of treatments and unit dose, depends on the protection or effect desired.

Precise amounts of the pharmaceutical composition also depend on the judgment of the practitioner and are particular to each individual. Factors affecting the dose include the physical and clinical state of the patient, the route of administration, the intended goal of treatment (e.g., alleviation of symptoms versus cure) and the potency, stability, and toxicity of the particular therapeutic substance.

Various combinations of the compounds of the disclosure and/or other traditional therapeutics for Schistosoma infection. For example, a first therapeutic compound or agent is “A” and a second therapeutic compound or agent is “B” can be combined and/or excluded, in a non-limiting manner as follows:

| | | | | |
|---|---|---|---|---|
| A/B/A | B/A/B | B/B/A | A/A/B | A/B/B |
| B/A/A | A/B/B/B | B/A/B/B | B/B/B/A | B/B/A/B |
| A/A/B/B | A/B/A/B | A/B/B/A | B/B/A/A | B/A/B/A |
| B/A/A/B | A/A/A/B | B/A/A/A | A/B/A/A | A/A/B/A. |

Administration of the therapeutic compounds or agents to a patient will follow general protocols for the administration of such compounds, taking into account the toxicity, if any, of the therapy. It is expected that the treatment cycles would be repeated as necessary. It also is contemplated that various standard therapies, as well as surgical intervention, may be applied in combination with the described therapy.

### II. CHEMICAL DEFINITIONS

Various chemical definitions related to such compounds are provided as follows.

As used herein, “predominantly one enantiomer” means that the compound contains at least 85% of one enantiomer, or more preferably at least 90% of one enantiomer, or even more preferably at least 95% of one enantiomer, or most preferably at least 99% of one enantiomer. Similarly, the phrase “substantially free from other optical isomers” means that the composition contains at most 5% of another enantiomer or diastereomer, more preferably 2% of another enantiomer or diastereomer, and most preferably 1% of another enantiomer or diastereomer.

As used herein, the term “water soluble” means that the compound dissolves in water at least to the extent of 0.010 mole/liter or is classified as soluble according to literature precedence.

Ar— can be aryl, substituted aryl, fused aryl, heterocyclic and heteroaryl, optionally substituted heterocyclic and heteroaryl, wherein said aryl, heterocyclic and heteroaryl groups can be optionally substituted by one or more R₄ groups. Unless otherwise indicated, as used herein, the term “aryl” includes an organic radical derived from an aromatic hydrocarbon by removal of one hydrogen, such as phenyl (Ph), naphthyl, indenyl, indanyl and fluorenyl. “Aryl” encompasses fused ring groups wherein at least one ring is aromatic. Unless otherwise indicated, as used herein, “heteroaryl” refers to aromatic groups containing one or more heteroatoms, preferably from one to three heteroatoms, selected from O, S and N. A multicyclic group containing one or more heteroatoms wherein at least one ring of the group is aromatic is a “heteroaryl” group.

The heteroaryl groups of this invention can also include ring systems substituted with one or more R₄ groups. Examples of heteroaryl groups are pyridinyl, pyridazinyl, imidazolyl, pyrimidinyl, pyrazolyl, triazolyl, pyrazinyl, quinolyl, tetrazolyl, furyl, thienyl, isoxazolyl, thiazolyl, oxazolyl, isothiazolyl, pyrrolyl, indolyl, benzimidazolyl, benzofuranyl, cinnolinyl, indazolyl, indolizinyl, phthalazinyl, triazinyl, 1,2,4-trizainyl, 1,3,5-triazinyl, isoindolyl, 1-oxoisoindolyl, purinyl, oxadiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, 1-[(1H-indol-3 -yl)methyl] and azaindolyl.

Examples of non-aromatic heterocyclic groups represented by Ar are aziridinyl, azetidinyl, pyrrolidinyl, piperidinyl, azepinyl, piperazinyl, tetrahydrofuranyl, tetrahydrothienyl, tetrahydropyranyl, tetrahydrothiopyranyl, morpholino and thiomorpholino.

R₄ can be alkyl, branched alkyl, substituted alkyl, halogen (F, Cl, Br, I), halogenated methyl (—CF₃, —CHF₂ and the like.), —CN, O-alkyl, halogenated O-alkyl (i.e. —OCF₃), —OH, NO₂, CO₂H, —CN, —CF₃, CO₂—R₁, CONH₂, CONHR₁, CON(R₁)₂, —NH₂, —NHR₁, —N(R₁)₂, —NHC(O)R₁.

Various groups are described herein as substituted or unsubstituted (i.e., optionally substituted). Optionally substituted groups may include one or more substituents independently selected from: halogen, nitro, cyano, hydroxy, amino, mercapto, formyl, carboxy, oxo, carbamoyl, substituted or unsubstituted alkyl, substituted or unsubstituted heteroalkyl, alkoxy, alkylthio, alkylamino, (alkyl)₂amino, alkylsulfinyl, alkylsulfonyl, arylsulfonyl, substituted or unsubstituted cycloalkyl, substituted or unsubstituted heterocyclyl (e.g., indole or indazole), substituted or unsubstituted aryl, and substituted or unsubstituted heteroaryl. In certain aspects the optional substituents may be further substituted with one or more substituents independently selected from: halogen, nitro, cyano, hydroxy, amino, mercapto, formyl, carboxy, carbamoyl, unsubstituted alkyl, unsubstituted heteroalkyl, alkoxy, alkylthio, alkylamino, (alkyl)₂amino, alkylsulfinyl, alkylsulfonyl, arylsulfonyl, unsubstituted cycloalkyl, unsubstituted heterocyclyl, unsubstituted aryl, or unsubstituted heteroaryl. Exemplary optional substituents include, but are not limited to: —OH, oxo (═O), —Cl, —F, Br, C₁₋₄alkyl, phenyl, benzyl, —NH₂, —NH(C₁₋₄alkyl), —N(C₁₋₄alkyl)₂, —NO₂, —S(C₁₋₄alkyl), —SO₂(C₁₋₄alkyl), —CO₂(C₁₋₄alkyl), and —O(C₁₋₄alkyl). The substitution can be at 1, 2, 3, 4, 5, 6, 7, 8, or more positions and present at position 1, 2, 3, 4, 5, 6, 7, and/or 8.

The term “pharmaceutically acceptable salts,” as used herein, refers to salts of compounds of this invention that are substantially non-toxic to living organisms. Typical pharmaceutically acceptable salts include those salts prepared by reaction of a compound of this invention with an inorganic or organic acid, or an organic base, depending on the substituents present on the compounds of the invention.

Non-limiting examples of inorganic acids which may be used to prepare pharmaceutically acceptable salts include: hydrochloric acid, phosphoric acid, sulfuric acid, hydrobromic acid, hydroiodic acid, phosphorous acid, and the like. Examples of organic acids which may be used to prepare pharmaceutically acceptable salts include: aliphatic mono- and dicarboxylic acids, such as oxalic acid, carbonic acid, citric acid, succinic acid, phenyl-heteroatom-substituted alkanoic acids, aliphatic and aromatic sulfuric acids, and the like. Pharmaceutically acceptable salts prepared from inorganic or organic acids thus include hydrochloride, hydrobromide, nitrate, sulfate, pyrosulfate, bisulfate, sulfite, bisulfate, phosphate, monohydrogenphosphate, dihydrogenphosphate, metaphosphate, pyrophosphate, hydroiodide, hydro fluoride, acetate, propionate, formate, oxalate, citrate, lactate, p-toluenesulfonate, methanesulfonate, maleate, and the like.

Suitable pharmaceutically acceptable salts may also be formed by reacting the agents of the invention with an organic base such as methylamine, ethylamine, ethanolamine, lysine, ornithine, and the like. Pharmaceutically acceptable salts include the salts formed between carboxylate or sulfonate groups found on some of the compounds of this invention and inorganic cations, such as sodium, potassium, ammonium, or calcium, or such organic cations as isopropylammonium, trimethylammonium, tetramethylammonium, and imidazolium.

Pharmaceutically acceptable salts of the compounds of Formula I include the acid or base addition salts thereof. All three reactions are typically carried out in solution. The resulting salt may precipitate out and be collected by filtration or may be recovered by evaporation of the solvent. The degree of ionization in the resulting salt may vary from completely ionized to almost non-ionized. Suitable non-toxic, acid-addition pharmaceutically acceptable salts include, but are not limited to, the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mandelates mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, salicylate, saccharate, stearate, succinate, sulfonate, stannate, tartrate, tosylate, trifluoroacetate and xinofoate salts.

Suitable non-toxic, base-addition pharmaceutically acceptable salts include, but are not limited to, the aluminium, arginine, benzathine, calcium, choline, diethylamine, diolamine, glycine, lysine, magnesium, meglumine, olamine, potassium, sodium, tromethamine and zinc salts. For a review on suitable salts, see Handbook of Pharmaceutical Salts: Properties, Selection, and Use by Stahl and Wermuth (Wiley-VCH, 2002).

Included within the scope of the present invention are all stereoisomers, geometric isomers and tautomeric forms of the compounds of Formula I, including compounds exhibiting more than one type of isomerism, and mixtures of one or more thereof.

The present invention includes all pharmaceutically acceptable isotopically-labelled compounds of Formula I wherein one or more atoms are replaced by atoms having the same atomic number, but an atomic mass or mass number different from the atomic mass or mass number which predominates in nature.

It should be recognized that the particular anion or cation forming a part of any salt of this invention is not critical, so long as the salt, as a whole, is pharmacologically acceptable.

Additional examples of pharmaceutically acceptable salts and their methods of preparation and use are presented in Handbook of Pharmaceutical Salts: Properties, Selection and Use (2002), which is incorporated herein by reference.

An “isomer” of a compound one is a separate compound in which each molecule contains the same constituent atoms as the compound one, but where the configuration of those atoms in three dimensions differs. Unless otherwise specified, the compounds described herein are meant to encompass their isomers as well. A “stereoisomer” is an isomer in which the same atoms are bonded to the same other atoms, but where the configuration of those atoms in three dimensions differs. “Enantiomers” are stereoisomers that are mirror images of each other, like left and right hands. “Diastereomers” are stereoisomers that are not enantiomers.

### III. SYNTHESIS OF COMPOUNDS OF FORMULA I

The compounds of the Formula I may be prepared by the methods described in example 1, together with synthetic methods known in the art of organic chemistry, or modifications and derivatizations that are familiar to those of ordinary skill in the art.

Preferred methods include, but are not limited to, those described in example 1. During any of the following synthetic sequences, it may be necessary and/or desirable to protect sensitive or reactive groups on any of the molecules concerned. This can be achieved by means of conventional protecting groups, such as those described in T. W. Greene, Protective Groups in Organic Chemistry, John Wiley & Sons, 1981; and T. W. Greene and P. G. M. Wuts,

Protective Groups in Organic Chemistry, John Wiley & Sons, 1991, which are hereby incorporated by reference.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Synthesis and Anti-Schistosomal Activity of Compounds Having a Chemical Structure of Formula I

The compounds of the Formula I may be prepared by the methods described below, together with synthetic methods known in the art of organic chemistry, or modifications and derivatizations that are familiar to those of ordinary skill in the art.

Preferred methods include, but are not limited to, those described below. During any of the following synthetic sequences, it may be necessary and/or desirable to protect sensitive or reactive groups on any of the molecules concerned. This can be achieved by means of conventional protecting groups, such as those described in T. W. Greene, Protective Groups in Organic Chemistry, John Wiley & Sons, 1981; and T. W. Greene and P. G. M. Wuts, Protective Groups in Organic Chemistry, John Wiley & Sons, 1991, which are hereby incorporated by reference.

Compounds of formula I or their pharmaceutically acceptable salts, can be prepared according to the following reaction Scheme 1 below. Isolation and purification of the products is accomplished by standard procedures, which are known to a chemist of ordinary skill. The following schemes and examples are exemplary of the processes for making compounds of formula I, wherein R₁=CH₂-1H-indol-3-yl)methyl, R₂=3-trifluoromethylbenzyl, and R₃=NO₂. It is to be understood, however, that the invention, as fully described herein and as recited in the claims, is not intended to be limited by the details of the following examples.

Referring to Scheme 1, alkylation of tert-butyl 3-cyanopyrrolidine-1-carboxylate 1 with benzyl bromide 2 in the presence of a suitable base such as LDA or KOt-Bu, in solvents such as THF, ACN or DMF, at temperatures ranging from −78° C. to room temperature provided the desired benzyl alkylated compound 3. Reduction of nitrile 3 with Raney-Nickel in ethanol under an atmosphere of H₂ to provided the desired amine 4. The amine 4 was coupled under Buchwald-Hartwig amidation conditions with the bromide 5 to form aniline 6 in 55% yield. Deprotection of the TBDMS group was achieved with TBAF in 80% yield, producing the desired alcohol 7. Deprotection of the t-butyl carbamate group was accomplished with BF₃OEt₂, which afforded secondary amine 8. Finally, reductive amination with an aldehyde such as 1H-indole-3-carbaldehyde, in the presence of a suitable reducing agent such as NaHB(OAc)₃, in solvents such as DCM, DCE and/or trifluroethanol (TFE), at temperatures raning from −20° C. to 50° C., preferably at room temperature, resulted in the formation of the desired compound 9 (CIDD-0149830).

Schistosomiasis screening methods Strains of S. mansoni, S. haematobium, and S. japonicum were maintained by passage through species-specific snail intermediate hosts (Biomphlaria glabrata, Bulinus truncatus, and Oncomelania hupensis, respectively) and Golden Syrian Hamsters as a definitive host. Definitive host animals were sacrificed in accordance with IACUC protocol (UTHSCSA IACUC Protocol #08039) by intraperitoneal injection using Fatal-Plus (Butler Animal Health, Ohio), a sodium pentobarbital solution, with 10% heparin added. The adult parasites were obtained by perfusion as previously described using 0.9% saline containing EDTA.¹ Derivatives were solubilized in DMSO and diluted to working concentration of 50 mM and added directly to each well of a 24-well plate within 2-24 hours after harvesting adult schistosomes from the hamsters at a final concentration of 143 μM. Each analog was tested in triplicate. DMSO, oxamniquine (OXA, the parent drug known to kill only S. mansoni), or Hycanthone (HC, a carcinogen known to kill S. mansoni and S. haematobium) were used as controls as needed. Drugs were incubated with the worms at 37° C., 5% CO₂ for 45 minutes, mimicking physiological conditions. The worms were washed with plain media 3 times to remove any residual derivatives. Worms were then incubated in culture media as previously described for a period of 10-14 days. On day 14, the assay gives % worms alive as an efficacy data point. For comparison, Oxaminiquine scores 50% alive in this assay. The compounds were first tested on S. mansoni, and a few of the more active analogs were tested on S. haematobium and S. japonicum.

### SYNTHETIC EXAMPLES

General procedures. All operations were carried out at room or ambient temperature, that is, in the range of 18-25° C.; evaporation of solvent was carried out using a rotary evaporator under reduced pressure with a bath of up to 50° C.; reactions were monitored by thin layer chromatography (tlc) and reaction times are given for illustration only. Unless otherwise indicated all reactions were conducted in standard commercially available glassware using standard synthetic chemistry methods and setup. All air- and moisture-sensitive reactions were performed under nitrogen atmosphere with dried solvents and glassware under anhydrous conditions. Starting materials and reagents were commercial compounds of the highest purity available and were used without purification (See list of specific reagents below). Solvents used for reactions were indicated as of commercial dry or extra-dry or analytical grade. Analytical thin layer chromatography was performed on aluminium plates coated with Merck Kieselgel 60F254 and visualized by UV irradiation (254 nm) or by staining with a solution of potassium permanganate. Flash column chromatography was performed on Biotage Isolera One 2.2 using commercial columns that were pre-packed with Merck Kieselgel 60 (230-400 mesh) silica gel. Final compounds for biological testing are all ≥95% purity as determined by HPLC-MS and ¹H NMR. ¹H NMR experiments were recorded on Agilent DD2 400 MHz spectrometers at ambient temperature. Samples were dissolved and prepared in deuterated solvents (CDCl₃, CD₃OD and DMSOd₆) with residual solvents being used as the internal standard in all cases. All deuterated solvent peaks were corrected to the standard chemical shifts (CDCl₃, dH=7.26 ppm; CD₃OD, dH=3.31 ppm; DMSOd₆, dH=2.50 ppm). Spectra were all manually integrated after automatic baseline correction. Chemical shifts (d) are given in parts per million (ppm), and coupling constants (J) are given in Hertz (Hz). The proton spectra are reported as follows: d (multiplicity, coupling constant J, number of protons). The following abbreviations were used to explain the multiplicities: app=apparent, b=broad, d=doublet, dd=doublet of doublets, ddd=doublet of doublet of doublets, dddd=doublet of doublet of doublet of doublets, m=multiplet, s=singlet, t=triplet. All samples were analyzed on Agilent 1290 series HPLC system comprised of binary pumps, degasser and UV detector, equipped with an auto-sampler that is coupled with Agilent 6150 mass spectrometer. Purity was determined via UV detection with a bandwidth of 170 nm in the range from 230-400 nm. The general LC parameters were as follows: Column-Zorbax Eclipse Plus C18, size 2.1×50 mm; Solvent A: 0.10% formic acid in water, Solvent B: 0.00% formic acid in acetonitrile; Flow rate—0.7 mL/min; Gradient: 5% B to 95% B in 5 min and hold at 95% B for 2 min; UV detector—channel 1=254 nm, channel 2=254 nm. Mass detector Agilent Jet Stream-Electron Ionization (AJS-ES).

The following abbreviations are used:

THF: tetrahydrofuran

DCM or CH₂Cl₂: dichloromethane

DCE: dichloroethane

NaHCO₃: sodium bicarbonate

HCl: hydrogen chloride

MgSO₄: magnesium sulfate

Na₂SO₄: sodium sulfate

DME: dimethoxyethane

n-BuLi: n-butyllithium

DMF: dimethylformamide

DMSO: dimethylsulfoxide

Et₂O: diethyl ether

MeOH: methanol

EtOAc: ethyl acetate

Procedures for Specific Examples.

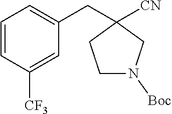 [SMILES: CC(C)(C)OC(=O)N1CCC(C#N)(Cc2cccc(C(F)(F)F)c2)C1]

tert-butyl 3-cyano-3-(3-(trifluoromethyl)benzyl)pyrrolidine-1-carboxylate (3): To a stirring, N₂ purged, solution of diisopropylamine (1.60 mL, 11.42 mmol) in THF (30.0 mL) in a flame dried 100 mL RBF at −78° C., was added a 2.5 M solution of n-BuLi (4.60 mL, 11.5 mmol) in hexanes dropwise over 5 min. After stirring for 10 min, was added tert-butyl 3-cyanopyrrolidine-1-carboxylate 1 (2.04 g, 10.41 mmol) in 10 mL of THF over 5 min by addition funnel. After stirring 1 h, was added 3-trifluoromethylbenzyl bromide (1.90 mL, 12.4 in 10 mL of THF over 6 min by addition funnel. After 1.5 h, reaction quenched upon addition of 20 mL of a saturated solution of NH₄OH, the layers were separated, and the aqueous portion was extracted with CH₂Cl₂ (3×15 mL). The combined organic portions were dried over Na₂SO₄ and concentrated under reduced pressure. Purification was accomplished by flash chromatography, eluting with gradients of 10% and 30% of EtOAc/Hexanes, collecting in 120 mL fractions. The product containing fractions (4-6) were concentrated under reduced pressure to obtain 2.84 g (77.1%) of nitrile 3 as a colorless oil. (mixture of rotamers) ¹H NMR (400 MHz, CDCl₃) ¹H NMR (400 MHz, DMSO) δ 7.68 (s, 2H), 7.64 (s, 2H), 3.58 (d, J=11.1 Hz, 1H), 3.55-3.45 (m, 1H), 3.40 (dd, J=11.4, 6.4 Hz, 1H), 3.36-3.28 (m, 26H), 3.23-3.08 (m, 2H), 2.20-2.11 (m, 2H), 1.40 (s, 9H). ¹⁹F NMR (376 MHz, DMSO) δ −61.20 (d, J=16.0 Hz). ESI-MS (m/z): 299.1 [M-C₄H₉+1].

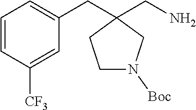 [SMILES: CC(C)(C)OC(=O)N1CCC(CN)(Cc2cccc(C(F)(F)F)c2)C1]

tert-butyl 3-(aminomethyl)-3-(3-(trifluoromethyl)benzyl)pyrrolidine-1-carboxylate (4): To a stirring solution of nitrile 3 (2.64 g, 7.44 mmol) in freshly distilled EtOH (13.0 mL) in a 50 mL RBF at room temperature, was added Raney-Nickel (41 mg). The flask was evacuated then back-filled with 1 atm of H₂ gas. After 16 h, the slurry filtered over celite, washing with CH₂Cl₂. The supernatant was collected and concentrated under reduced pressure. Purification was accomplished by flash chromatography, eluting with solvent gradient of 5% and 10% MeOH/CH₂Cl₂, collecting in 120 mL fractions. The product containing fractions (5-7) were concentrated under reduced pressure to obtain amine 4 (2.43 g, 95.4%) as a colorless oil. (mixture of rotamers) ¹H NMR (400 MHz, DMSO) δ 7.53 (s, 2H), 7.49 (s, 2H), 3.23-3.11 (m, 2H), 3.08-2.93 (m, 2H), 2.75 (s, 2H), 2.31 (s, 2H), 1.70-1.49 (m, 4H), 1.39-1.30 (m, 9H). ¹⁹F NMR (376 MHz, DMSO) δ −61.20 (d, J=16.0 Hz). ESI-MS (m/z): 359.2 [M+1].

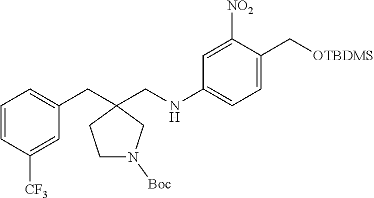 [SMILES: CC(C)(C)OC(=O)N1CCC(CNc2ccc(CO[Si](C)(C)C(C)(C)C)c([N+](=O)[O-])c2)(Cc2cccc(C(F)(F)F)c2)C1]

tert-butyl 3-(((4-(((tert-butyldimethylsilyl)oxy)methyl)-3-nitrophenyl)amino)methyl)-3-(3-(trifluoromethyl)benzyl)pyrrolidine-1-carboxylate (6): To a stirring solution of amine 4 (2.34 g, 6.54 mmol) in toluene (22.0 mL) at room temperature, added aryl bromide 5 (2.43 g, 7.02 mmol), Cs₂CO₃ (6.54 g, 20.0 mmol) and (±)-BINAP (206.1 mg, 0.331 mmol). A stream of N₂ gas was bubbled through the solution for 5 minutes, after which time Pd(OAc)₂ (73.6 mg, 0.328 mmol). The reaction was heated to reflux for 18 hours under positive N₂, cooled to room temperature, diluted with CH₂Cl₂, filtered over celite and the celite pad was washed with CH₂Cl₂. The resulting crude solution was concentrated under reduced pressure to yield a brown oil. Purification was accomplished by flash chromatography, eluting with gradients of 5% and 15% EtOAc/hexanes, collecting in 120 mL fractions. The product containing fractions (6-9) were concentrated under reduced pressure to obtain 2.23 g (54.7%) of aniline 6 as a red oil. ¹H NMR (400 MHz, CDCl₃) δ 7.63 (d, J=8.2 Hz, 1H), 7.51 (d, J=7.2 Hz, 1H), 7.41 (t, J=7.5 Hz, 1H), 7.37 (s, 1H), 7.31 (d, J=7.4 Hz, 1H), 6.85 (dd, J=8.4, 1.2 Hz, 1H), 4.98 (s, 2H), 3.45 (s, 2H), 3.36 (s, 2H), 3.02 (s, 2H), 2.88 (s, 2H), 1.89 (td, J=13.4, 6.7 Hz, 1H), 1.82 (td, J=13.3, 6.7 Hz, 1H), 1.47 (s, 8H), 0.95 (s, 8H), 0.12 (s, 5H). ¹⁹F NMR (376 MHz, CDCl₃) δ −62.71. ESI-MS (m/z): 646.3 [M+23].

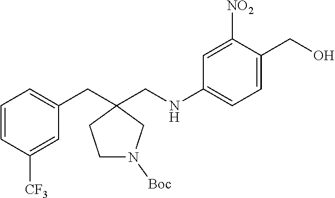 [SMILES: CC(C)(C)OC(=O)N1CCC(CNc2ccc(CO)c([N+](=O)[O-])c2)(Cc2cccc(C(F)(F)F)c2)C1]

tert-butyl 3-(((4-(hydroxymethyl)-3-nitrophenyl)amino)methyl)-3-(3-(trifluoromethyl)benzyl)pyrrolidine-1-carboxylate (7): To a stirring solution of silyl ether 6 (2.19 g, 1.45 mmol) in THF (10.0 mL) in a flame dried 100 mL RBF at 0° C., was added a 1.0 M Tetrabutylammonium Fluoride (4.2 mL) solution in THF dropwise over 5 min and the resulting reaction was allowed to warm to room temperature. After 2 h, 20 mL of brine and 15 mL of EtOAc were added to quench the reaction. Layers were separated, and the aqueous portion was extracted with EtOAc (2×20 mL). The combined organic layers were dried over Na₂SO₄ and concentrated under reduced pressure. Purification was accomplished by flash chromatography, eluting with solvent gradients of 20% and 40% of EtOAc/Hexanes, collecting in 120 mL fractions. The product containing fractions (3-6) were concentrated under reduced pressure to obtain 1.45 g (81.1%) of alcohol 7 as an orange oil. ¹H NMR (400 MHz, CDCl₃) δ 7.51 (d, J=7.1 Hz, 1H), 7.43-7.37 (m, 2H), 7.34 (s, 1H), 7.28 (d, J=7.9 Hz, 1H), 7.21 (d, J=2.4 Hz, 1H), 6.80 (dd, J=8.3, 2.4 Hz, 1H), 4.76 (s, 2H), 3.58-3.40 (m, 2H), 3.36 (d, J=11.2 Hz, 1H), 3.32-3.15 (m, 1H), 3.01 (s, 2H), 2.93-2.80 (m, 2H), 1.93-1.75 (m, J=19.6, 12.8, 6.5 Hz, 2H), 1.44 (d, J=8.8 Hz, 9H). ESI-MS (m/z): 454.2 [M-C₄H₉+1].

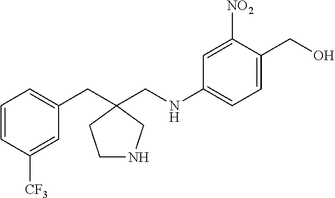 [SMILES: O=[N+]([O-])c1cc(NCC2(Cc3cccc(C(F)(F)F)c3)CCNC2)ccc1CO]

(2-nitro-4-(((3-(3-(trifluoromethyl)benzyl)pyrrolidin-3-yl)methyl)amino)phenyl)methanol (8): To a stirring solution of tertbutyl carbamate 7 (1.04 g, 2.04 mmol) in CH₂Cl₂ (9.0 mL) in a flame dried 50 mL RBF at −10° C., apparatus was flushed with N₂ before boron trifluoride etherate (1.00 mL, 8.10 mmol) was added one portion and the resulting reaction was allowed to slowly warm to room temperature. After 4 h, 15 mL of a saturated NaHCO₃ solution was added quench the reaction. The layers were separated, and the aqueous portion was extracted with a 3:1 mixture of isopropanol and CHCl₃ (6×15 mL). The combined organic layers were dried over Na₂SO₄ and concentrated under reduced pressure. Purification was accomplished by flash chromatography, eluting with solvent gradients of 5% and 20% 1 M NH₄OH in MeOH/CH₂Cl₂, collecting in 22 mL fractions. The product containing fractions (2-4) were concentrated under reduced pressure to obtain 246.5 mg (29.5%) of amine 8 as an orange oil. ¹H NMR (400 MHz, cd₃od) δ 7.42 (d, J=8.6 Hz, 1H), 7.25-7.12 (m, 6H), 6.92 (dd, J=8.5, 2.4 Hz, 1H), 4.74 (s, 2H), 2.99-2.88 (m, J=22.9 Hz, 3H), 2.87-2.78 (m, J=17.6, 4.1 Hz, 2H), 2.74 (d, J=11.5 Hz, 1H), 1.90-1.77 (m, 1H), 1.76-1.60 (m, 1H). ¹⁹F NMR (376 MHz, DMSO) δ −63.45. ESI-MS (m/z): 410.2 [M+1].

The general reductive amination procedure used to prepare compounds like 9 from the corresponding amine 8, is described below.

To a stirring solution of amine (1 equiv.) in 1,2-DCE (0.1 M), added the appropriate aldehyde or ketone (1.5 equiv.). After stirring for 1 hour at room temperature, NaBH(OAc)₃ (2.5 equiv) was added. The reaction was allowed to stir for 12-24 hours, at which point, reaction was quenched upon addition of 1.0 ml of saturated NaHCO₃ and 1.0 mL of EtOAc. Layers were separated then the aqueous portion was extracted EtOAc (3×10 mL). Combined organic fractions, dried over Na₂SO₄, and concentrated under reduced pressure. Purification crude product was carried out via by flash chromatography to afford the desired compounds. The following compounds were prepared using the general method described above.

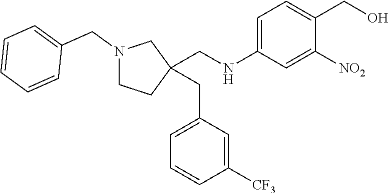 [SMILES: O=[N+]([O-])c1cc(NCC2(Cc3cccc(C(F)(F)F)c3)CCN(Cc3ccccc3)C2)ccc1CO]

(4-(((1-benzyl-3-(3-(trifluoromethyl)benzyl)pyrrolidin-3-yl)methyl)amino)-2-nitrophenyl)methanol (CIDD-0149831): Orange oil: 8.5 mg 48.3%. ¹H NMR (400 MHz, cd₃od) δ 7.95 (d, J=8.0 Hz, 2H), 7.51-7.23 (m, 5H), 7.20 (d, J=2.5 Hz, 1H), 6.90 (dd, J=8.5, 2.5 Hz, 1H), 4.75 (s, 2H), 3.74 17 (ABq, ΔV_{AB}=18.3 Hz, J_{AB}=12.6 Hz, 2H), 2.96 (dd, J=15.2, 5.2 Hz, 2H), 2.89 (d, J=15.7 Hz, 1H), 2.73 (d, J=13.1 Hz, 1H), 2.68 (d, J=10.9 Hz, 1H), 2.00-1.87 (m, 1H), 1.81-1.68 (m, 1H). ¹⁹F NMR (376 MHz, DMSO) δ −64.03. ESI-MS (m/z): 500.2 [M+1].

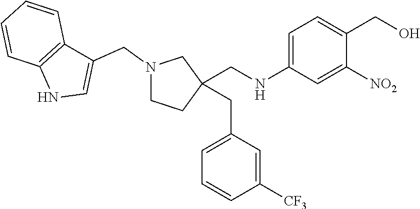 [SMILES: O=[N+]([O-])c1cc(NCC2(Cc3cccc(C(F)(F)F)c3)CCN(Cc3c[nH]c4ccccc34)C2)ccc1CO]

(4-(((1-((1H-indol-3-yl)methyl)-3-(3-(trifluoromethyl)benzyl)pyrrolidin-3-yl)methyl)amino)-2-nitrophenyl)methanol (CIDD-0149830): Orange oil: 7.4 mg, 39.0%. ¹H NMR (400 MHz, cd₃od) δ 7.64 (d, J=7.8 Hz, 1H), 7.48 (d, J=11.6 Hz, 2H), 7.42 (d, J=8.5 Hz, 1H), 7.40-7.31 (m, 3H), 7.17 (d, J=2.4 Hz, 1H), 7.13 (d, J=8.1 Hz, 1H), 7.08 (t, J=7.0 Hz, 1H), 6.87 (dd, J=8.5, 2.5 Hz, 1H), 4.75 (m, 2H), 3.17 (ABq, Δ_{AB}=12.4 Hz, J_{AB}=13.6 Hz, 2H), 3.15 (s, 1H), 2.96 (s, 13H), 2.11-2.01 (m, 5H), 1.81-1.68 (m, 1H). ¹⁹F NMR (376 MHz, DMSO) δ −64.15. ESI-MS (m/z): 539.2 [M+1].

TABLE 1
The IUPAC names, CIDD#’s and lot #’s for individual compounds screened for anti-
schistosomal activity are illustrated below.

| | | | Lot Created | |
|---|---|---|---|---|
| CIDD# | Structure | IUPAC NAME | Date | Lot ID |
| CIDD- 0149831 | 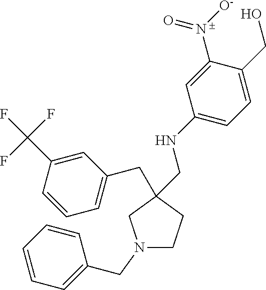 | (4-{[(1-benzyl-3-{[3- (trifluoromethyl)phenyl] methyl}pyrrolidin-3- yl)methyl]amino}-2- nitrophenyl)methanol | 2018 Jul. 18 | SM2018-83-91 |
| CIDD- 0149830 | 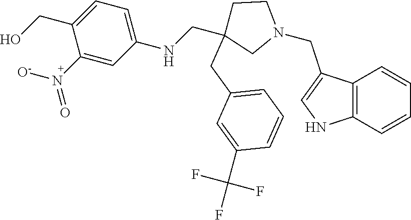 | {4-[({1-[(1H-indo1-3-yl) methyl]-3-{[3- (trifluoromethyl)phenyl] methyl}pyrrolidin-3- yl}methyl)amino]-2- nitrophenyl}methanol | 2018 Jul. 18 | SM2018-83-90 |
| CIDD- 0149829 | 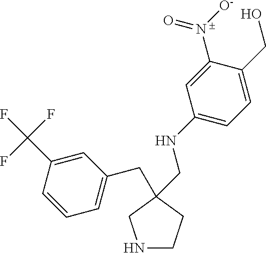 | (2-nitro-4-{[(3-{[3- (trifluoromethyl)phenyl] methyl}pyrrolidin-3- yl)methyl]amino}phenyl) methanol | 2018 Jul. 18 | SM2018-73-167 |

TABLE 2
Killing Data for selected compounds; Schistosoma worm killing activity.

| CIDD | Kill % S. m | Kill % S. h | Kill % S. j |
|---|---|---|---|
| CIDD-0149829 | NA | NA | NA |
| CIDD-0149830 | 100 | 100 | 100 |
| CIDD-0149831 | NA | NA | NA |
| CIDD-0150235 | 60 | NA | NA |
| CIDD-0150236 | 43 | NA | NA |
| CIDD-0150237 | 33 | NA | NA |
| CIDD-0150238 | 6 | NA | NA |
| CIDD-0150239 | 36 | NA | NA |
| CIDD-0150240 | 20 | NA | NA |
| CIDD-0150241 | 33 | NA | NA |
| CIDD-0150242 | 56 | NA | NA |
| CIDD-0150302 | 80 | 20 | 40 |
| CIDD-0150303 | 100 | 40 | 100 |
| CIDD-0150326 | 3.3 | NA | NA |
| CIDD-0150327 | 3.3 | NA | NA |
| CIDD-0150328 | 16 | NA | NA |
| CIDD-0150329 | 40 | NA | NA |
| CIDD-0150330 | 10 | NA | NA |
| CIDD-0150331 | 10 | NA | NA |
| CIDD-0150332 | 17 | NA | NA |
| CIDD-0150333 | 20 | NA | NA |
| CIDD-0150334 | 47 | NA | NA |
| CIDD-0150603 | 100 | 78.6 | NA |
| CIDD-0150604 | 90 | 92.9 | NA |
| CIDD-0150605 | 13 | NA | NA |
| CIDD-0150606 | 100 | 85.7 | NA |
| CIDD-0150607 | 50 | NA | NA |
| CIDD-0150608 | 93.3 | 71.4 | NA |
| CIDD-0150609 | 16.6 | NA | NA |
| CIDD-0150610 | 100 | 100 | 100 |

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit, and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope, and concept of the invention as defined by the appended claims.

1. A compound having a chemical structure of Formula I 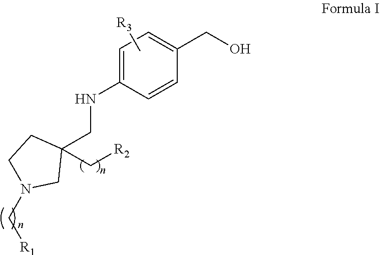
wherein n is 0 or 1; R₁ and R₂ are independently selected from H, alkyl, branched alkyl, substituted alkyl, aryl, and —CH₂—Ar; R₃ is F, Cl, Br, I, —NO₂, CO₂H, —CN, —CF₃, CO₂—R1, CONH₂, CONHR₁, or CON(R₁)₂.

2. The compound of claim 1, wherein R₁=CH₂-1H-indol-3-yl)methyl, R₂=3-trifluoromethylbenzyl, and R₃=NO₂.

3. The compound of claim 1, wherein R₁=CH₂C₆H₅, R₂=3-trifluoromethylbenzyl, and R₃=NO₂.

4. The compound of claim 1, wherein R₁=H, R₂=3-trifluoromethylbenzyl, and R₃=NO₂

5. A composition comprising an effective amount of a compound having a chemical structure of Formula I, or a pharmaceutically acceptable salt or solvate thereof.

6. A method of treating a Schistosoma infection, the method comprising administering an effective amount of a compound having a chemical structure of Formula I or a pharmaceutically acceptable salt or solvate thereof, to a subject in need thereof.

7. The method of claim 6, further comprising administering an effective amount of praziquantel.

8. The method of claim 6 or 7, wherein the subject is human.

9. The method of any one of claims 6 to 8, wherein the Schistosoma infection is an infection by one or more of Schistosoma mansoni, Schistosoma haematobium or Schistosoma japonicum.
